(19) United States
(12) Patent Application Publication
(10) Pub. No.: US 2008/0202515 A1
(43) Pub. Date: Aug. 28, 2008

(54) Inhalation Device

(75) Inventors: Peter D. Hodson, Berkshire (GB); Stephen J. Howgill, Leicestershire (GB); Graham R. Purkins, Berkshire (GB)

Correspondence Address: 3M INNOVATIVE PROPERTIES COMPANY, PO BOX 33427, ST. PAUL, MN, 55133-3427, US

(73) Assignee: 3M INNOVATIVE PROPERTIES COMPANY, ST. PAUL MINNESOTA, MN (US)

(21) Appl. No.: 11/721,991

(22) PCT Filed: Dec. 12, 2005
(86) PCT No.: PCT/US05/45021

§ 371 Date: Jun. 18, 2007

(30) Foreign Application Priority Data
Dec. 23, 2004 (GB) 0428169.7

(51) Int. Cl. A61M 15/00

(52) U.S. Cl. 128/203.21

(57) ABSTRACT

An inhalation device (1) comprising two or more hermetically sealed receptacles (39), each receptacle containing a single dose of a pressurized formulation comprising a pharmaceutically active ingredient and a liquefied aerosol propellant consisting of HFA 134a, HFA 227 or a mixture thereof and wherein at least a portion (39a) of each receptacle is perforable.

This invention relates generally to inhalation devices comprising two or more hermetically sealed receptacles, each holding a single pre-metered dose of pressurized liquefied propellant-based formulation.

Pressurized metered dose inhalers have been used for over forty years for the treatment of asthma and other respiratory conditions. Pressurized metered dose inhalers comprise a container filled with many doses of propellant-based formulation, together with a metering valve for dispensing individual metered doses upon demand. One of the disadvantages of conventional metered dose inhalers is the difficulty in providing a low number of doses (e.g. less than thirty) that is appropriate for some therapy regimes. Furthermore, although dry powder inhalation devices or liquid nasal devices, which sometimes provide individual doses of formulation, are commercially available and although a single dose inhalation device including gases such as carbon dioxide, oxygen or nitrogen has been proposed (see U.S. Pat. No. 4,137,914 published in 1979), to date no commercially viable inhalation device providing individual pre-metered pressurized doses has been proposed or commercialized.

### SUMMARY OF THE INVENTION

There is a need to provide a commercially viable inhalation device including individual pre-metered pressurized doses suitable for delivering a pharmaceutically active ingredient to the lung.

We have found that through the use of hermetically sealed receptacles, each containing a single dose of a pressurized formulation comprising a pharmaceutically active ingredient and a liquefied aerosol propellant consisting of 1,1,1,2-tetrafluoroethane (HFA 134a), 1,1,1,2,3,3,3-heptafluoropropane (HFA 227) or a mixture thereof (as the sole propellant component) and wherein at least a portion of the receptacle is perforable (i.e. capable of being perforated), an inhalation device is provided in which upon actuation of the device and thus perforation of a receptacle, an aerosol is generated allowing for delivery of the pharmaceutically active ingredient to the lung. Moreover, the generated aerosol for inhalation advantageously comprises fine droplets of liquefied propellant together with pharmaceutically active ingredient allowing for reliable delivery and transport of the active ingredient to the patient's lungs.

Thus the present invention provides an inhalation device comprising two or more hermetically sealed receptacles, each containing a single dose of a pressurized formulation comprising a pharmaceutically active ingredient and a liquefied aerosol propellant consisting of HFA 134a, HFA 227 or a mixture thereof and wherein at least a portion of each receptacle is perforable.

In the previously proposed pressurized single dose devices (see U.S. Pat. No. 4,137,914 and U.S. Pat. No. 6,062,213) including e.g. compressed gases such as carbon dioxide, oxygen or nitrogen, upon actuation (or activation) of said system, the gas immediately vaporizes thus generating a “hard” aerosol with just active ingredient with the devices being unsuitable for delivery of medicament to the lung or the correct portion thereof. In the devices proposed in U.S. Pat. No. 4,137,914 it has been recognized that much of the medicament will simply be deposited on to the internal surface of the mouthpiece (if in fact the dose is able to pass through the disclosed capillary tube prior to occlusion thereof. In the devices disclosed in U.S. Pat. No. 6,062,213, the device is to be provided with sufficiently high vapor pressure at room temperature such that proactive delivery means vaporize thoroughly upon activation (providing a hard aerosol with just active ingredient) and similar to the devices disclosed in US'914 much of the medicament will simply be immediately deposited on surfaces of the device and/or retained within the device.

The receptacles of the devices described herein preferably have relatively low internal pressure within the receptacle at ambient temperature (22° C.), e.g. at most 7 atmospheres pressure absolute. This is advantageous inter alia in terms of cost effectiveness and ease in manufacture, in that the receptacle need not be spheroid in shape (a form typically necessary for containers having a high internal pressure). Receptacles also desirably have an internal volume of less than 0.3 ml. The volume of the pressurized formulation is typically 150 μl at most.

Devices in accordance with the invention preferably further comprise a firing pin for perforating the receptacle, more preferably two or more firing pins, wherein each receptacle is in operative association with a firing pin. In other words, in preferred embodiments the device includes a corresponding number of firing pins to the number of receptacles, so that each receptacle is associated with its own operatively associated firing pin. This is advantageous in that for the perforation of each receptacle a fresh firing pin is used and thus any potential problems of blunting and/or occlusion upon a repetitive use of a single firing pin may be avoided. Also through the application of one-use firing pins, devices described herein can be manufactured more efficiently and effectively due to more favorable construction and material selection considerations. Each firing pin comprises a channel having an opening at both ends; more desirably the channel is defined by an internal surface of the firing pin. Also, favorably the at least perforable portion of the receptacle may be provided as a substantially planar portion to help avoid deflection or skidding of the firing pin used to perforate the receptacle. Such perforation is preferably rapid, in order to avoid or minimize unwanted aerosol leakage during perforation. In particular to provide a reliable and rapid firing force for perforating the receptacle (independent of any force provided by the patient), upon actuation of the device the receptacle to-be-fired and firing pin are desirably mutually displaced, through the use of a triggering mechanism that is released upon actuation to act on the receptacle and/or firing pin as the case may be, to cause the firing pin to perforate the receptacle at said at least perforable portion and to cause aerosol formulation to pass through said channel from the first end to the second end of the channel and through to the patient. The term “mutually displaced” means that the receptacle is displaced towards the firing pin, or the firing pin is displaced towards the receptacle, or both the receptacle and firing pin are displaced towards each other. To avoid deflection by or skidding of the firing pin during actuation, preferably the firing pin is held fixed at the time of actuation and the receptacle to-be-fired is displaced towards the firing pin, for example through the use of a triggering mechanism including a mechanically or pneumatically loaded impactor that is released upon actuation to act on the receptacle displacing it towards the firing pin to cause the firing pin to perforate the receptacle at said at least perforable portion. It is preferable that the device is arranged such that upon perforation of said at least perforable portion of the receptacle by the firing pin, the first end of the channel of the firing pin passes into a liquid portion of the pressurized formulation. To facilitate temporary sealing and/or to minimize unwanted leakage between the firing pin and receptacle upon perforation, it may be advantageous that the outer surface of the portion of the firing pin (e.g. the tip thereof that passes into the receptacle is conical in shape.

To minimize occlusion within the channel of the firing pin the channel is preferably not provided in the form of a capillary tube. The internal surface of the said firing pin channel may be arranged to provide an expansion chamber, i.e. a chamber through which the expanding aerosol passes after its release from the perforated receptacle and before its subsequent passage through a more restrictive region such as a spray break-up orifice. To further minimize the potential for occlusion, the internal surface of said channel may advantageously be generally conical from the first end to the second end. Desirably the channel of the firing pin extends generally along a single axis. Typically the outermost surface of the firing pin (e.g. the outermost portion of the tip of the firing pin) is provided with a piercer capable of perforating the at least perforable portion of the receptacle. In order to avoid or minimize unwanted channel obstruction caused by any deformation of the firing pin and/or undesirable aerosol leakage upon perforation, it has been found advantageous to slightly offset the inlet of the channel from said outermost surface of the firing pin. Thus in preferred embodiments, the first end of the channel of the firing pin is set back from said outermost surface and positioned adjacent to said piercer.

Devices described herein, desirably further comprise a carrier wherein the receptacles are mounted on the carrier. The carrier may be in the form of a carousel, wherein the receptacles are mounted onto the carousel such that the at least perforable portion of each receptacle is positioned radially outwardly. Favorably, in those embodiments including firing pins in operative association with receptacles, the firing pins may also be mounted on the carrier, for example such that the first end of the channel of each individual firing pin is positioned towards the at least perforable portion of its associated receptacle. For re-filling the device with new receptacles (and in preferred embodiments their associated firing pins), a portion of the device comprising the carrier and mounted receptacles (and in preferred embodiments their associated firings pin) may advantageously be reversibly removable from the device. This portion may be suitably provided in the form of a cassette. Favorably devices described herein may be provided in the form of a kit-of-parts comprising a body portion and a cassette including receptacles (and in preferred embodiments their associated firing pins) and optionally one or more such cassettes as replacement cassettes.

Devices described herein suitably further comprise a patient outlet adapted for insertion into a patient's mouth or nose (nostril or nostrils) having a passageway. Devices described herein desirably further comprise an advancing mechanism, in particular a mechanical advancing mechanism, wherein the advancing mechanism is arranged such that prior to actuation of the device, the receptacle to be fired (and in preferred embodiments its associated firing pin) is (are) advanced to a registered position in alignment with the patient-outlet in preparation for actuation, more desirably, the advancing mechanism is arranged, such that an advancement of any subsequent receptacle to the registered position is prohibited until the preceding advanced receptacle has been actuated (i.e. fired). Advantageously, the device further comprises a patient-outlet cover and said cover is coupled to the advancing mechanism, such that upon opening the cover the receptacle to be fired (and in preferred embodiments its associated firing pin) is (are) advanced to the registered position, more particularly such cover is coupled to the advancing mechanism, such that if the receptacle advanced to the registered position upon opening of the outlet cover has not been actuated (i.e. fired) then upon closing the outlet cover said receptacle (and if applicable its associated firing pin) is (are) returned to its pre-advanced position so that when the cover is subsequently opened said receptacle (if applicable together with its associated firing pin) is (are) advanced again to the registered position. Herein the perforation of the receptacle, preferably via the use of a firing pin and a triggering mechanism as described herein, to cause aerosol formulation to pass out of the receptacle and through to the patient is generally referred to in abbreviation as actuating the receptacle or firing the receptacle.

Devices described herein may be actuated either manually (e.g. by pressing a button or lever) or by inhaling (i.e. by breath-actuation). Breath-actuation is preferred. For example, the delivery of a pharmaceutically active ingredient for treatment of a systemic disease will tend to require the aerosol to penetrate the deep lung (e.g. to the alveolar regions). This requires good coordination of dose release with the early part of the patient's inspiratory manoeuvre. Breath-actuation provides a reliable way of ensuring such coordination, particularly for patients using an inhaler for the first time or for patients using an inhaler infrequently.

The dependent claims define further embodiments of the invention.

The invention, its embodiments and further advantages will be described in the following with reference to the following drawings or figures.

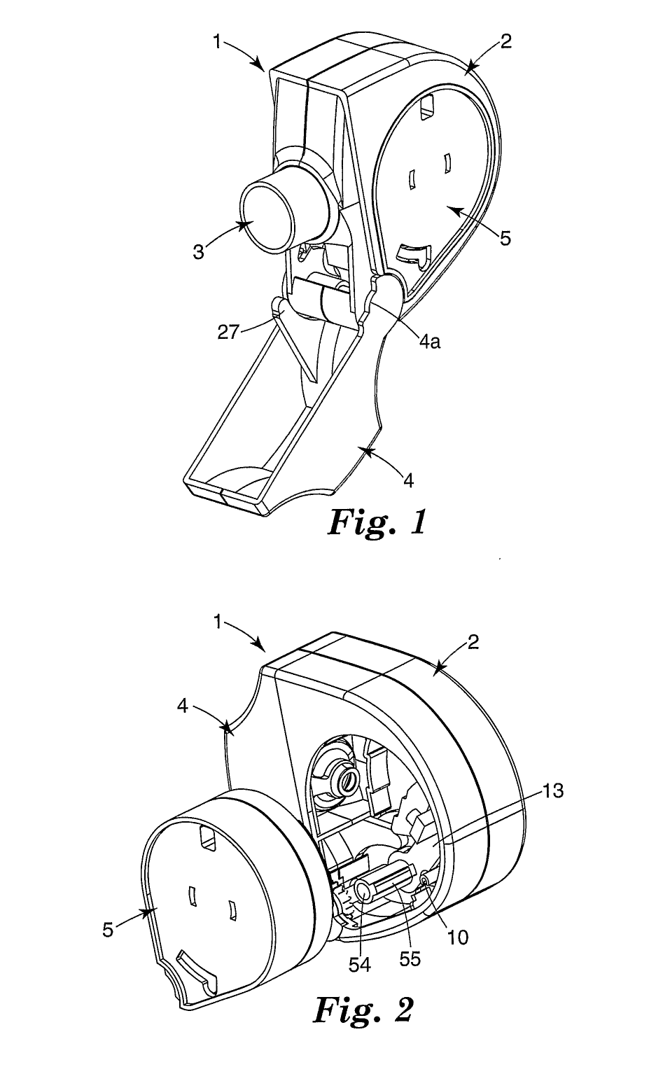

FIGS. 1 to 3 show perspective views of an exemplary embodiment of a device in accordance with the present invention.

FIGS. 4, 5 and FIG. 13 represent exploded diagrams of particular sub-assemblies of the embodiment shown in FIGS. 1 to 3.

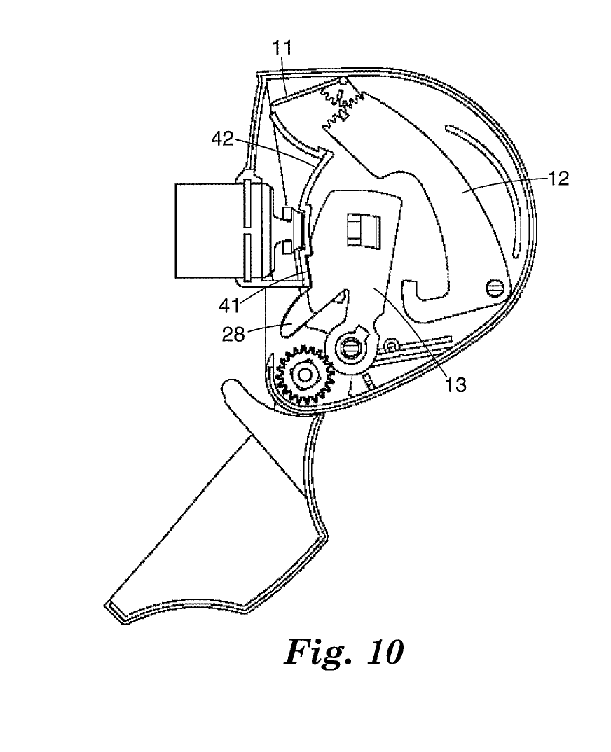

FIGS. 6 to 9 represent schematic interior views of the embodiment shown in FIGS. 1 to 3 in its rest position, primed position, in an intermediate position of actuation and in its fired position, respectively, while FIG. 10 represents schematic interior view of the opposite side of the embodiment in its fired position.

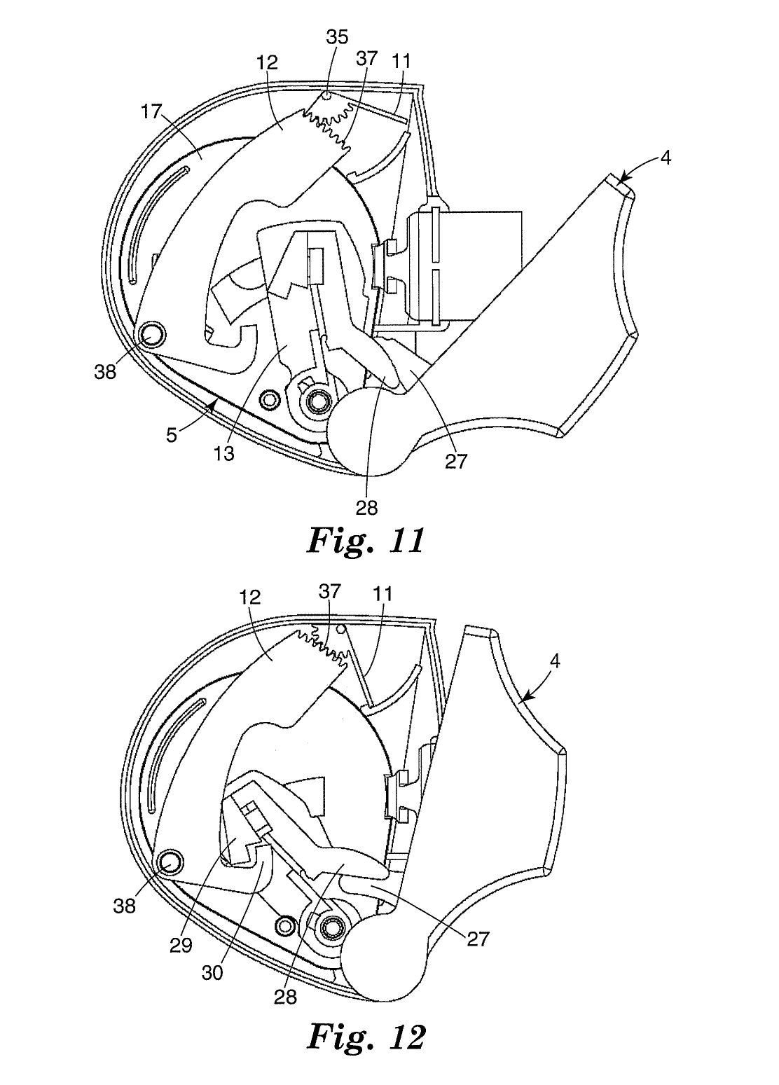

FIGS. 11 and 12 represent schematic interior views of the embodiment shown in FIGS. 1 to 3 in two intermediate positions upon return to its rest position after actuation.

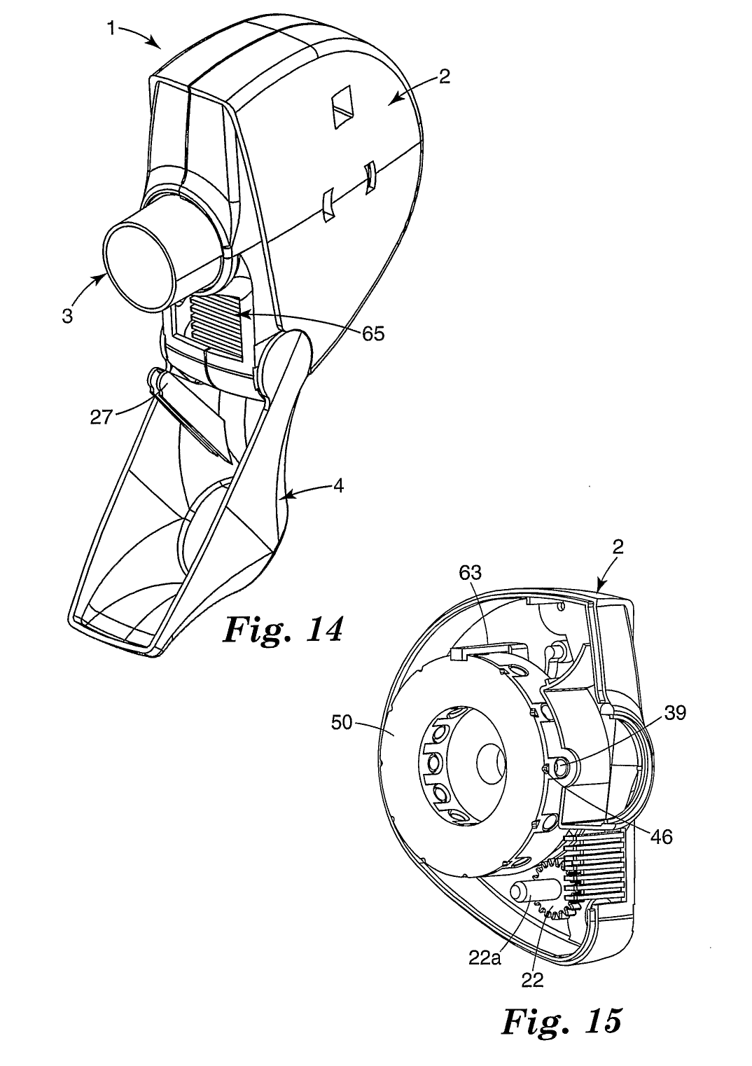

FIG. 14 represents a perspective view of another exemplary embodiment of a device in accordance with the present invention.

FIG. 15 represents a perspective view of a portion of the exemplary embodiment shown in FIG. 14.

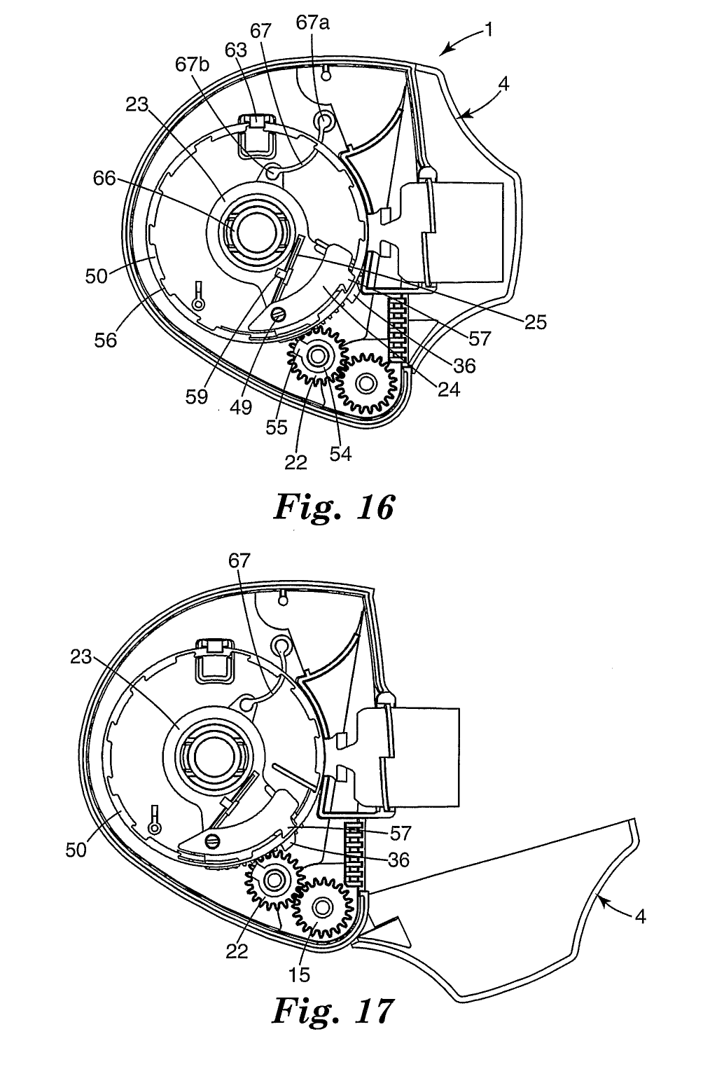

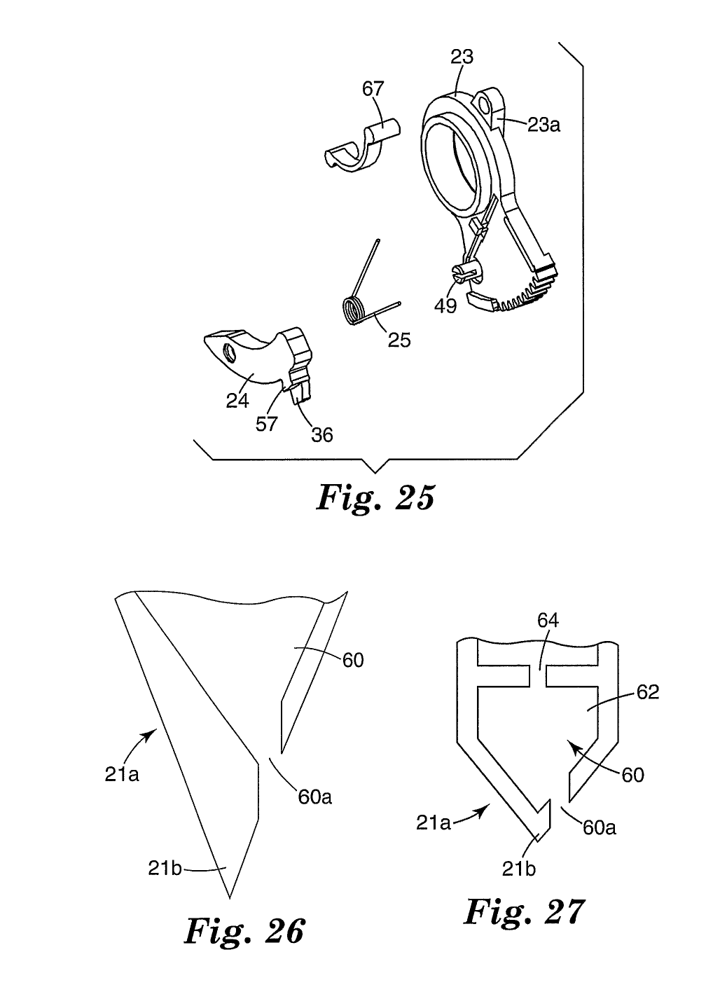

FIGS. 16 to 24 represent schematic interior views of the embodiment shown in FIG. 14 at various stages of the operation of a dose advancing mechanism, while FIG. 25 represents an exploded diagram of a sub-assembly of the dose advancing mechanism.

FIG. 26 represents an enlarged, schematic cross-section of a preferred formation of a portion of a tip of a firing pin for both the exemplary embodiments shown in FIGS. 1 to 13 and 14 to 25, respectively.

FIG. 27 represents an enlarged, schematic cross-section of an alternative preferred formation of a portion of a tip of a firing pin for both the exemplary embodiments shown in FIGS. 1 to 13 and 14 to 25, respectively.

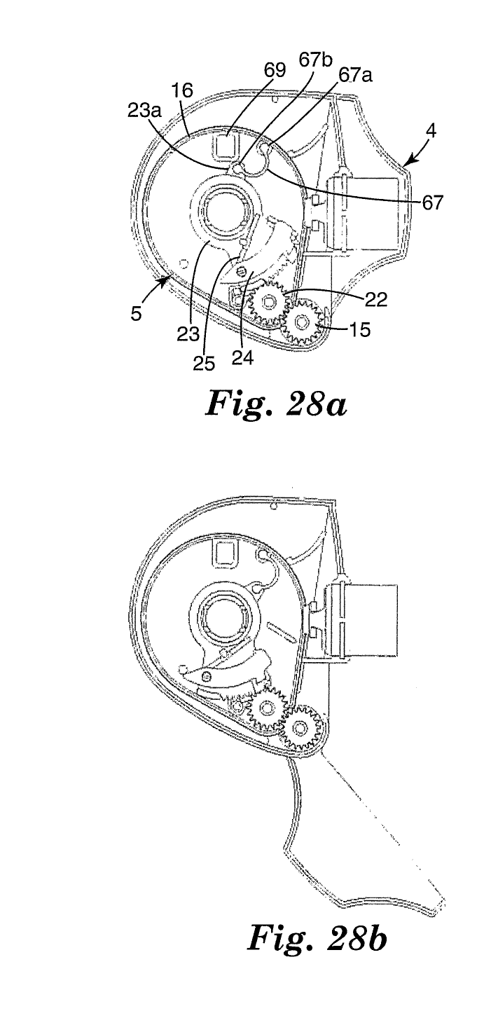

FIG. 28a and b represent schematic interior views of a portion of yet another exemplary embodiment.

It is to be understood that not all the Figures are provided at the same scale.

It is also to be understood that the present invention covers all combinations of suitable, favorable, particular, desirable, advantageous, and preferred aspects of the invention described herein.

FIGS. 1 to 13 illustrate a first exemplary embodiment in accordance with the present invention. This exemplary embodiment is a refillable breath-actuated device.

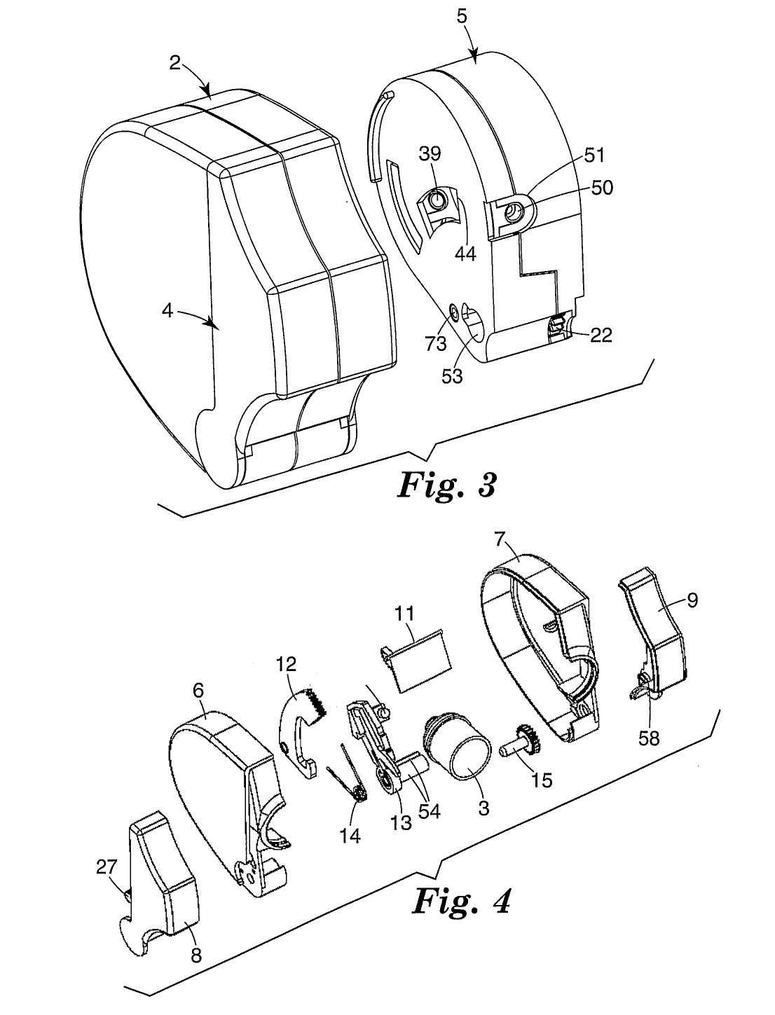

Referring to FIGS. 1 to 3, the exemplary device (1) comprises a reusable body portion (2) and a replaceable cassette (5) containing a plurality of receptacles (39). The device comprises an outlet (3) adapted for insertion into a patient's mouth. Alternatively the outlet may be adapted for insertion into a patient's nasal cavity (e.g. for delivery of a pharmaceutically active ingredient to nasal mucosa). In the following the outlet adapted for insertion into a patient's mouth or nasal cavity or cavities will generally be referred to under the term patient-outlet. The device may include a hinged outlet cover (4) that can be used by the patient to cover the patient-outlet (3) when the inhaler is not in use, thereby protecting the device against dirt and moisture ingress. The outlet cover (4) is shown in its open position in FIG. 1 and in its closed position in FIGS. 2 and 3. The device may be provided with a tactile and/or audible feedback for the patient indicating that the device or outlet is fully opened or fully closed, for example by providing small pips (not shown) on the outlet cover (4) and small indentations (not shown) on the body portion (2) which give a “click” interaction when the patient opens or shuts the outlet cover. Suitably the patient-outlet (3) is a push-fit into the body portion (2), so that it can be detached for the patient to wash it.

FIGS. 2 and 3 show the receptacle-containing cassette (5) removed from the body portion (2) of the exemplary device (1), e.g. for replacement with a fresh cassette. Removal and replacement of cassettes typically involve simple sliding motions, the cassette being held in place by conventional “snap-fit” features (not shown). Referring to FIG. 3, the cassette (5) typically includes a number of openings, such as a mounting hole (73), a cassette driving opening (53) and a cassette impact opening (44). When the cassette (5) is inserted into the body portion (4), components located within the body portion (see FIG. 2), such as a C-shaped mounting post (10) and an impactor (13) having a head (43) and a hollow shaft (54) with a cam (55) (the function of these components will be explained below), can be received by the cassette through the respective openings of the cassette. The cassette also includes a front opening (51) (see FIG. 3) that is positioned towards the patient-outlet (3) facing a spray inlet (52) of the patient-outlet when the cassette is inserted into the body portion. In FIG. 3, visible through the front opening (51) is a carousel (50) containing the receptacles, while visible through the impactor opening (44) is a bottom surface of another receptacle (39).

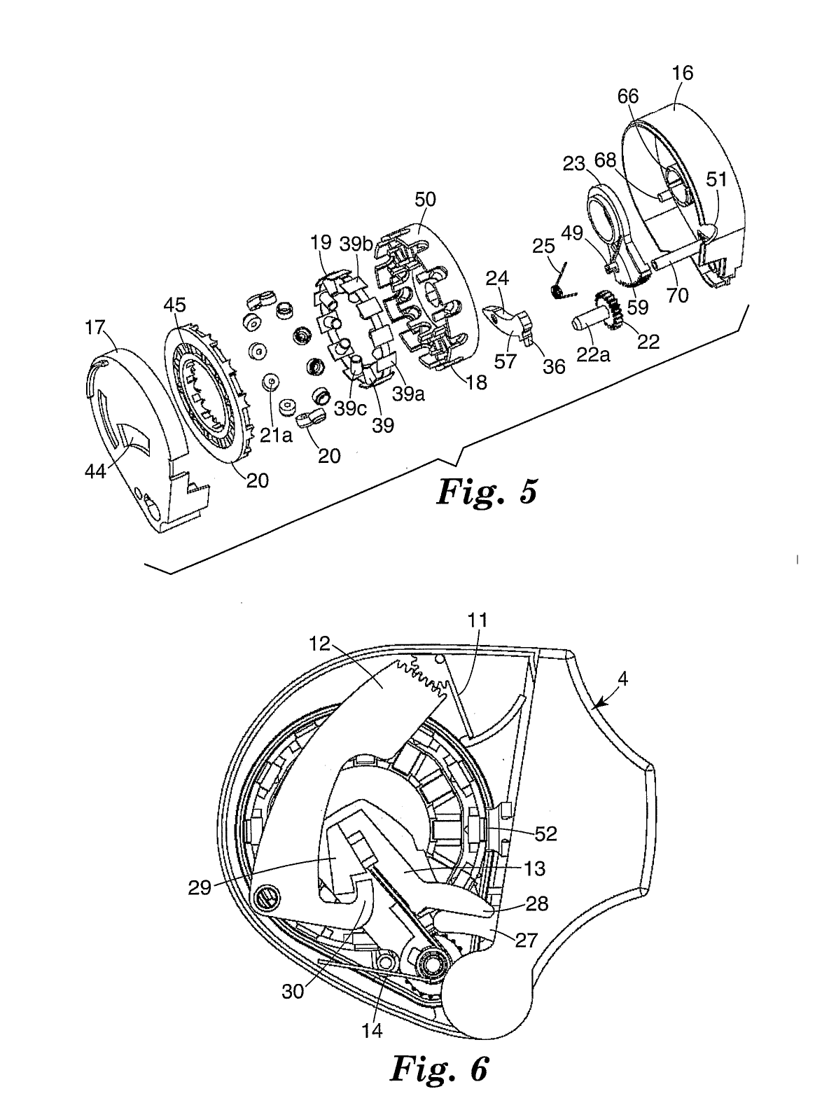

The profile (4a in FIG. 1) of the outlet cover (4) is suitably configured such that the cassette (5) can only be removed or replaced when the cover (4) is in its closed position, and thus the device is in its rest position (explained in more detail below), in order to ensure that the impactor (13) is not in a position (e.g. its fired position) where it would cause an obstruction and possible damage. Also, when the impactor (13) is in its fired position, the cam (55) on the driving shaft (54) of the impactor is in a position where it cannot pass through the cassette driving opening (53, see FIG. 5) and thus the cam also helps to facilitate the retention of the cassette (5) in the body portion (2) when the impactor is in its fired position.

Referring to FIG. 5, which represents an exploded diagram of the cassette (5) of the exemplary device (1), the cassette includes outer (16) and inner (17) casing halves. As can be recognized from FIG. 5, the exemplary device includes twelve hermetically sealed receptacles (39), each comprising at least a perforable portion (39a). The receptacles may be provided on an elongated strip (19) and mounted in a carousel (50), whereby the carousel comprising two housing halves (18,20).

The perforable portion of each receptacle may advantageously be in the form of a foil (see e.g. component 39b in FIG. 5). Such a foil may be e.g. laser welded (as described in our co-pending application GB 0418738 filed Aug. 23, 2004, incorporated herein by reference) onto a receptacle body (see e.g. component 39c in FIG. 5). The receptacle or any of its components, e.g. a foil or a receptacle body, may be made of metal such as stainless steel or aluminum. The perforable portion of the receptacle may be provided as a substantially planar portion, so as to minimize skidding or deflection of a firing pin (discussed below) as the firing pin contacts the portion. The at least perforable portion of the receptacle suitably has a thickness of at most 250 μm. For robustness of the receptacle, a thickness of at least 25 μm is favorable for the at least perforable portion. For enhanced robustness a thickness of at least 38 μm is desirable, more desirably of at least 50 μm. To further facilitate access to the receptacle e.g. through piercing, a thickness of at most 150 μm is desirable, more desirably of at most 100 μm, most desirably of at most 75 μm. As can be appreciated from the exemplary device shown in FIGS. 1 to 13, in order to provide a desirable compact device, it is advantageous to arrange the receptacles, for example onto a carousel within the device, in the general form of a circle with the at least perforable portions of the receptacles facing radial outwardly from the center point of the circle.

Each receptacle contains a single dose of a pressurized formulation comprising a pharmaceutically active ingredient and a liquefied aerosol propellant consisting of HFA 134a, HFA 227 or a mixture thereof. Receptacles used in inhalation devices described herein are desirably free of elastomeric seals and diaphragms and/or dispensing valves, which is advantageous in avoiding leaking during storage as well as any ingress of air or moisture from the outside environment and/or any undesirable interaction with seal and/or diaphragm materials. The receptacles are desirably appropriately dimensioned (e.g. having a low or a minimum amount of head space) for containing a single dose of HFA-134a and/or HFA-227 based medicinal formulation. To accommodate a single dose of such a pharmaceutically active aerosol formulation, the receptacle desirably has an internal volume of less than 0.3 ml, more desirably 0.2 ml or less, even more desirably 0.15 ml or less, most desirably about 0.15 ml. Suitably the internal volume of the receptacle is at least 0.1 ml. As mentioned above the volume of pressurized formulation is typically 150 μl at most, and more desirably about 100 μl at most, and most desirably about 80 μl at most. Typically the pressurized formulation has a volume of at least about 25 μl, and more desirably at least about 40 μl and most desirably at least about 50 μl. Also mentioned above, the internal pressure within the receptacle is desirably at most 7 atmospheres absolute at ambient temperature, more desirably at most about 6.5 atmospheres absolute, even more desirably from about 3 to about 6.5 atmospheres absolute, most desirably from about 4 to about 6.5 atmospheres absolute.

Returning to the exemplary embodiment shown in FIGS. 1 to 13, each receptacle (39) is in operative association with a firing pin (21). Thus the exemplary device includes twelve individual firing pins. As can be appreciated from the exemplary embodiment, the firing pins may be suitably mounted onto a carrier, e.g. onto the carousel (50), such that the perforable portion (39a) of each receptacle (39) is positioned facing towards its associated firing pin (21), in particular to a piercer capable of perforating the perforable portion provided on the tip (21a) of the firing pin.

Each firing pin desirably has an internal surface defining a channel, wherein the channel has an opening at its first end, positioned towards the receptacle at least at the time of actuation, and an opening at its second end, positioned towards the patient outlet at least at the time of actuation. In order to provide compact devices, it is desirable that the channel of the firing pin defines substantially a single axis and the outlet passageway extends substantially along or substantially parallel to that axis at the time of actuation of the device. To minimize any potential for occlusion and/or to provide favorable aerosol characteristics, advantageously the channel may be generally conical from the first end to the second end.

The outermost surface of the firing pin is suitably provided with a piercer, e.g. in the form of a sharp point. Desirably the outer surface of the tip of the firing pin, positioned towards the receptacle at least at the time of actuation, is conical in shape. Referring to FIG. 26 showing schematically an enlargement of a portion of a tip (21a) of a firing pin, the first end (60a) of the channel (60) is advantageously slightly set back from the outermost surface of the firing pin and positioned adjacent to said piercer (21b). FIG. 27 shows an enlargement of an alternative tip (21a), in which the internal surface defining the channel (60) is arranged as to provide an expansion chamber (62) with a spray break-up orifice (64).

The firing pin or the tip thereto is suitably made of a material, e.g. a material comprising a metal or a polymeric material, allowing perforation of the at least perforable portion of the receptacle. Surprisingly, it has been found that injection molded polymeric firing pins, such as those comprising polybutyleneterephthalate, acetal and/or polycarbonate, can be used to effectively perforate perforable portions of hermetically sealed receptacles, even when said portions are formed from metal (e.g. stainless steel or aluminum), e.g. metallic foil (such as 50 μm thick stainless steel foil). Alternative forms of piercer, such as oblique-cut stainless steel points are also suitable.

Again making reference to the exemplary embodiment and FIG. 5, the cassette also comprises a gear (22), an indexing yoke (23), an indexing arm (24), and a torsion spring (25). The function of these components, which form part of the dose advancing mechanism, will become clear in the description below. Referring to FIG. 4 which represents an exploded diagram of the components of the body portion of the exemplary embodiment, the body portion includes besides the removable patient-outlet (3), the outlet cover (4) formed from two components (8,9) and the impactor (13), a housing made of two outer casing halves (6, 7), a vane (11), a rocker (12) an impactor spring (14) and a drive gear (15). The impactor (13) and the impactor spring (14), together with the vane (11) and rocker (12), form part of the triggering mechanism of the exemplary device, while the drive gear (15) forms part of the dose advancing mechanism. The function of these components will be explained below.

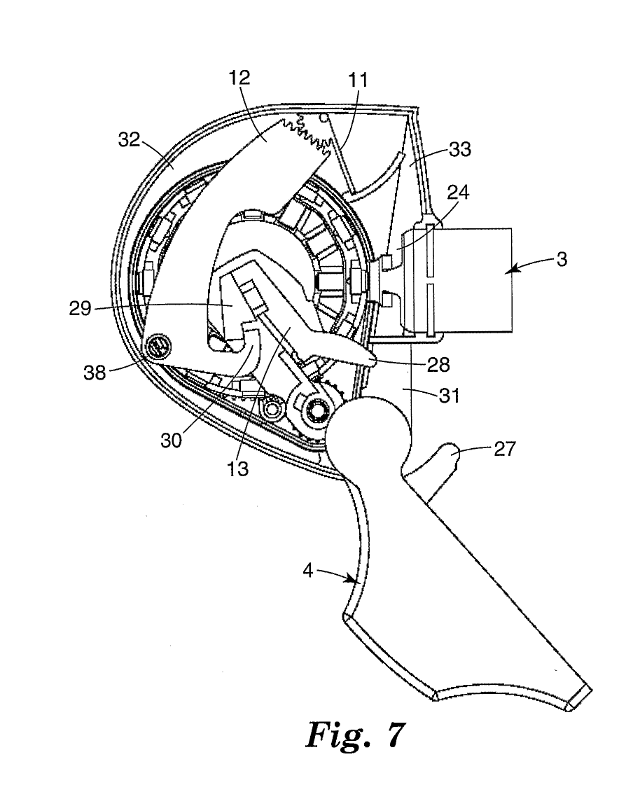

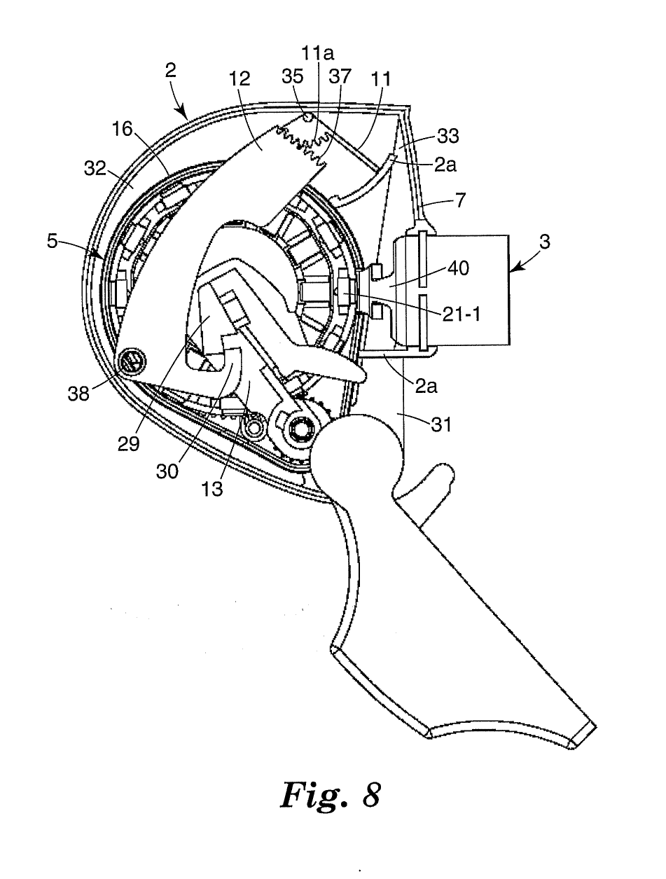

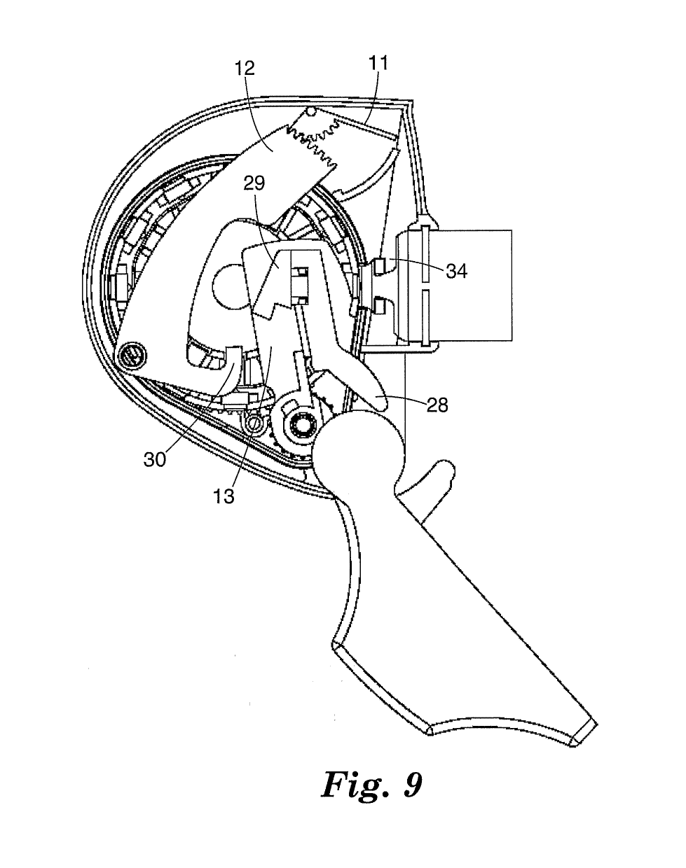

The actuation or triggering of the exemplary device will be described with reference to FIGS. 6 to 9 which provide a schematic view of the interior of the device at its rest (and closed) position (FIG. 6), its primed position (FIG. 7), an intermediate position of actuation (FIG. 8) and its final fired position (FIG. 9). In these figures for ease in viewing the position of the receptacles and firing pins during various stages of actuation, the inner casing (17) of the cassette (5) as well as the inner housing half (20) of the carousel (50) have been omitted from the figures.

As can be seen from FIG. 6, in its rest and closed position, the outlet cover is closed with a reset arm (27) extending from the outlet cover (4) resting against, and thereby preventing from moving, an arm (28) of the impactor (13). A protrusion (29) on the impactor (13) sits within a large hook (30) at the end of the rocker (12), although the protrusion (29) is not in contact with the hook.

When the patient opens the cover (4) (as shown in FIG. 7), the reset arm (27) is pulled away from the arm (28) of the impactor (13). The impactor (13) moves thereby slightly forward (from left to right, i.e. clockwise, in FIG. 7) under the influence of the impactor spring (14: omitted from FIGS. 7 to 9 for the sake of clarity, but recognizable from FIG. 6) until the impactor protrusion (29) comes to rest against the large hook (30) on the rocker (12). In this primed position of the device, the triggering mechanism of the device, which comprises the vane (11), rocker (12), impactor (13), and impactor spring (14), is stable, due to the slight “over centre” direction of action of the force of the impactor spring (14) about the pivot point (38) of the rocker (12). The triggering mechanism can however readily be released by inhalation through the patient-outlet (3), due to “mechanical advantage” provided by preferable geometries of the vane (11) and the rocker (12).

Referring to FIG. 8, when the patient places the patient-outlet (3) in their mouth and inhales, air is drawn into the device from an air inlet (31) through an internal airflow channel (32), via the vane (11), through an internal airflow opening (33) and through air inlets (34) into the patient outlet (3). This causes the vane (11) to start to rise (counter-clockwise in FIG. 8), as it rotates about its pivotal axis (35). Close-fitting walls on all sides (referring to FIG. 8 e.g. the outer casing halves (6,7 (6 is not visible)) of the body portion (2), the outer casing halves (16,17 (17 is not visible) of the cassette (5), and walls (2a and 2b) provided within the body portion) ensure that the flow of air does not significantly by-pass the vane (11) at this stage. As the vane (11) rotates around its pivotal axis (35), gear teeth (11a) on the vane (11) cause the gear teeth (37) on the rocker (12) to move, causing the rocker (12) to rotate clockwise around its own pivot (38). As the rocker rotates, its large hook (30) starts to slide off the protrusion (29) on the impactor (13).

As the patient continues to inhale, the vane (11) continues to rotate (counter-clockwise) further (as shown in FIG. 9), and the rocker (12) in turn rotates sufficiently for its large hook (30) to release the protrusion (29) on the impactor (13). Driven by the impactor spring (14), the impactor (13) freely rotates (clockwise in FIG. 9) at high speed such that the impactor (13) strikes the receptacle (39-1) that is aligned with the patient-outlet (3). In particular as the head (43; not clearly visible in FIG. 9) of the impactor (13) strikes the base of the receptacle (39-1), the receptacle is displaced (pushed) towards its associated firing pin, such the top surface of the receptacle comprising the perforable portion strikes against its associated firing pin (21a), causing the latter to puncture the perforable portion. As the receptacle is punctured, the outer surface of the firing pin (the tip thereof makes a temporary seal with the lid, whilst the dose of medicament formulation inside the receptacle (39-1) passes through the channel of the firing pin (21-1) and out through the inlet (52) and passageway of the patient-outlet (3) to the patient. The patient-outlet may include an inner spacer portion (40) (between inlet (52 in FIG. 1) and the generally cylindrical outer portion (3a) of the patient-outlet) provided with air-inlets (34) to create an annular air-jacket around the emerging aerosol. The internal surface of the inner spacer portion (40) of the patient-inlet is desirably generally conical. Due to the characteristic positioning of the arm (28) of the impactor (13) in the fired position of the device, the arm (which might be advantageous brightly colored) may be suitably used as an indicator to appear in a window (26 in FIG. 4) in the device, thereby indicating to the patient that the mechanism has been triggered (i.e. that a dose has been released).

Referring to FIG. 10 showing a schematic interior view of the exemplary device in its fired position (i.e. as per FIG. 9) from the opposite side, the front edge (41) of the impactor (13) may be suitably shaped to match the contour of the internal front wall (42) against which the cassette rests. By virtue of these matching contours, the energy of the moving impactor (13) would be safely dissipated across a wide area if the inhaler were to be triggered without a cassette being present. However due to the presence of the air inlets (34) in the inner spacer portion (40) of the patient-outlet (3), air leakage through the inner spacer portion created by the lack of a cassette, should in fact bypass the vane (11) to such an extent that the triggering mechanism could not be released by inhalation in the absence of a cassette. However, it is possible that extremely rough handling of a primed but empty (i.e. no cassette loaded) device might release the triggering mechanism.

The resetting of the triggering mechanism of the exemplary device will be described with reference to FIGS. 11 and 12, which provide schematic views of the interior of the device in which the outlet cover is partially closed (FIG. 11) and the outlet cover is almost completely closed (FIG. 12). For the sake of clarity the impactor spring (14) has been omitted. In FIGS. 11 and 12 (unlike FIGS. 6 to 9) the inner casing half (17) of the cassette (5) has not been omitted, and thus the receptacles and firing pins are not visible. As the patient closes the outlet cover (4), the reset arm (27) of the cover moves towards the arm (28) of the impactor (13) (FIG. 11) and then pushes back the impactor (13) against the force of its spring (14; again omitted from FIGS. 11 & 12) (FIGS. 11 and 12). As the impactor (13) returns towards its rest position (as shown FIG. 6), its protrusion (29) bears against the rocker (12), causing it to rotate slightly counter-clockwise around its pivot (38). As it rotates, the rocker pulls the vane (11) shut again (FIG. 12) via the interaction of its gear teeth (37) with those on the vane. The upper surface of the reset arm (27) may be suitably profiled such that during the final part of the reset process (from the position shown in FIG. 12 to that shown in FIG. 6), the interaction of the reset arm (27) with the impactor (13) causes the forces to “go over centre”. In other words the force of the impactor spring (14) then tends to bias the impactor (13) and the outlet cover (4) slightly towards their respective “at rest” positions (as shown in FIG. 6). Finally, the impactor (13) moves slightly away from the rocker (12), thus “unloading” the rocker (12) and vane (11) mechanism at the rest position of the device.

Although not yet described it is to be appreciated that for the exemplary embodiment shown in FIGS. 1 to 13, upon opening and closing the outlet cover (4), a dose advancing mechanism including an indexing yoke (23), an indexing arm (24), a torsion spring (25), a gear (22) and a drive gear (15) moves appropriately the carousel (50) and thus the receptacle to-be-fired and its associated firing pin. Since the second exemplary embodiment incorporates a similar dose advancing mechanism, said mechanism and its operation will be described with reference to the second exemplary embodiment shown in FIGS. 14 to 25.

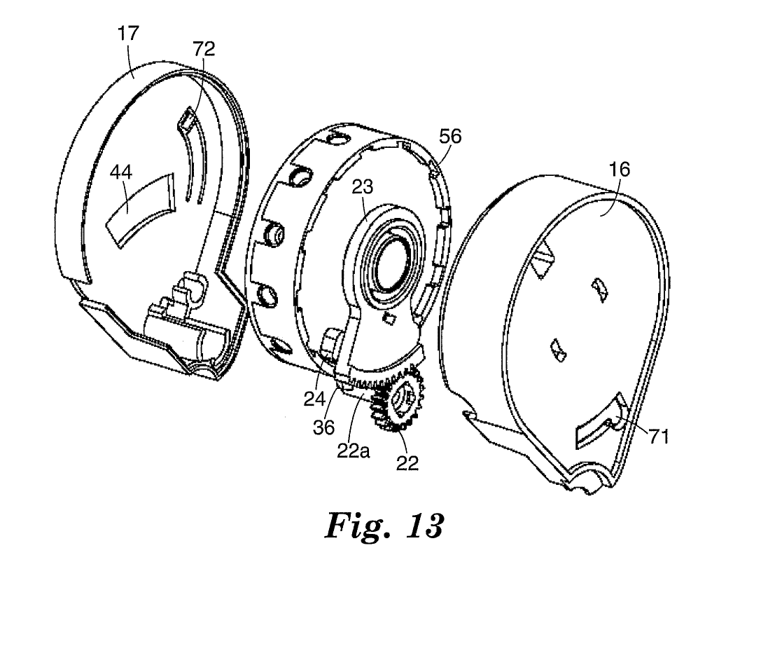

FIG. 13 shows further features of the first exemplary embodiment. Because the cassette (5) can be removed from the inhaler body portion (2) by the patient, and because its gear (22) is then exposed, tamper prevention means are provided to ensure that the patient does not move the carousel between doses, i.e. to a position where a receptacle (39) will be out of registration with the patient outlet (3) (in particular the inlet (52) to the patient-outlet). These tamper prevention means take the form of a small clip (not shown) that prevents movement of the indexing yoke (23) when the cassette (5) is out of the body portion (2). The clip is mounted on an integral spring mounting (71) that is cantilevered out from the wall of the outer case (16) of the cassette. Also molded onto the spring mounting (71) is a tall post (70 in FIG. 5). As the cassette (5) is pushed into the body portion (2), another, C-profiled, post (10 in FIG. 2) in the body portion (2) passes through an access hole (73 in FIG. 3) into the cassette (5), and meets the tall post (70) end-to-end. This causes the spring mounting (71) that carries the tall post (70) to deflect slightly, deflecting the clip (not shown) away from the indexing yoke (23), thereby allowing the yoke to rotate (as required during advancing) when the cassette is mounted into the body portion (2), but not when the cassette is outside (i.e. not mounted into) the body portion (2).

The first exemplary embodiment also includes components for centering the carousel (50). In particular referring to FIG. 13, the inner casing (17) is provided with an integral springy finger (72) that interacts with a series of recesses or dimples (45)—one for each receptacle—on the top surface of the carousel (see FIG. 5). These recesses (45) individually co-operating with the springy finger upon dose advancement provide the required degree of self-centering alignment or quantization of the carousel parking position. A tactile and/or audible feedback from the finger and recess arrangement may desirably provide an indication to the patient that the next dose has been advanced successfully.

FIGS. 14 to 25 illustrate a second exemplary embodiment in accordance with the present invention. This exemplary embodiment is a non-refillable breath-actuated device. This exemplary device is similar to the device shown in FIGS. 1 to 13 with a major difference being that the device is non-refillable and thus the device includes a body (2) that is fully enclosed.

Referring to FIG. 14 the device (1) comprises a body (2), a patient-outlet in the form of a mouthpiece (3), and a hinged outlet cover (4) provided with a reset arm (27). In this embodiment, the patient-outlet (3) is provided with a screw-fit with a definite stop position to facilitate good contact between the outlet and the carousel (50, not visible in FIG. 14) in which the receptacles and firing pins are mounted. A grill (65) may be provided across most of the width of the frontal air inlet (31) in order to e.g. prevent the intrusion of patient fingers. Although not shown in any of the FIGS. 14 to 25, this second exemplary embodiment includes like the first exemplary embodiment a triggering mechanism (comprising an impactor (13) (having a hollow impactor shaft (54) provided with a cam (55)), an impactor spring (14), a vane (11), and a rocker (12)) which is actuated by breath actuation, arranged and operated in the same manner as described above for the first exemplary embodiment. It is to be understood that the complete description concerning the triggering of the first exemplary embodiment applies to the second embodiment.

Referring to FIG. 15, which provides a perspective view of a portion of the device, the receptacle and firing pin-containing carousel (50) of the exemplary device is held into one half of the body (2) by a retaining clip (63). Molded onto the underside of the clip (63), but not visible in FIG. 15, is a protruding V-shaped pip, which co-operates with small recesses (e.g. 46) on the carousel (50), one recess associated with each receptacle (39) to provide an additional positive centering of the position of the carousel (50), and hence of each receptacle (39), after each dose advancement. Tactile and/or audible feedback from the pip and recess arrangement also desirably provides a signal to the patient that the next receptacle has been advanced successfully and thus is in position for actuation.

The dose advancing or indexing of the exemplary device will be described with reference to FIGS. 16 to 25 providing schematic views of a portion of the interior of the device at various stages of dose advancement (FIGS. 16 to 24) and an exploded diagram of a sub-assembly of the dose advancing mechanism (FIG. 25). For the sake of clarity the receptacles, firing pins and most of the carousel structure as well as most of the components forming part of the triggering mechanism have been omitted in FIGS. 16 to 24.

Advantageously, the dose advancing mechanism of the exemplary device advances the carousel to bring the next receptacle to-be-fired into its registered position (i.e. in alignment with the patient-outlet (and the inlet thereof), and thus ready for actuation, when the patient-outlet cover is opened, in particular on the condition that the previous receptacle was impacted and thus fired.

FIG. 16 shows an interior view of the exemplary device (1) in its rest position, with the outlet cover (4) closed. The receptacle and firing pin-containing carousel (50) is clipped onto a central boss (66) in the device, and is held in place by the retaining clip (63). Also mounted on the central boss (66), but free to rotate relative to the carousel (50), is an indexing yoke (23), the teeth of which are engaged with those of a gear (22). Clipped onto a peg (49) molded on the indexing yoke (23) sits an indexing arm (24). A torsion spring (25) is held between the indexing arm (24) and a spring stop (59) on the indexing yoke (23), such that the indexing arm (24) is biased outwardly (i.e. pivoted clockwise around peg (49) in FIG. 16), with its indexing tooth (57) engaged in one of a sequence of carousel advancement recesses (56) inside the rim of the carousel (50). One such recess (e.g. 56) corresponds to each of the twelve dose-containing receptacles in the carousel (50). The shapes of the indexing tooth (57) and of the carousel advancement recesses (56), and their relative positions and angles, are such that the torsion spring (25) tends to keep them biased into engagement in either rotational direction. A curved bias link (67) may be anchored to a point (67a) on the internal surface of the body (2) and to a point (67b) on an extension (23a) provided on the indexing yoke (23). The anchor points (67a, 67b) allow some rotational movement of the ends of the bias link (67) but are spaced so that the bias link is not loose. The gear (22) is provided with a shaft (22a). The shaft (22a) is inserted into the hollow impactor shaft (54) having a cam (55) of the impactor (13, not completely shown). The cam (55) will in certain situations (described in detail below) interact with a pawl (36) provided on the indexing arm (24). As mentioned above the teeth of the gear (22) are engaged with those of the indexing yoke (23). The teeth of the gear (22) are also engaged with the teeth of a drive gear (15). The drive gear is in turn engaged with a square peg (58, not visible (see FIG. 4 showing the square peg in the first exemplary embodiment)) provided on the internal surface of the outlet cover (4).

FIGS. 17 to 19 provide schematic representations of the interior of device when the outlet cover (4) is partially opened (at about 65°), almost completely open (at about 120°) and completely open (at about 130°).

Referring to FIG. 17, as the outlet cover (4) is opened, its square peg (58, not visible) drives a corresponding square keyway (15a, not visible) in the drive gear (15), causing both gears (15, 22) to rotate. The gear (22) in turn causes the indexing yoke (23) to rotate. As it does so, the distance between the two anchor points for the curved bias link (67) decreases slightly, so that the bias link (67) is forced to bend, thereby providing some resistance in opening the outlet cover.

The as-molded curvature of the bias link (67) ensures that it bends without tending to buckle as its ends move closer together. As the indexing yoke (23) and the indexing arm (24) rotate, the indexing tooth (57) pulls the carousel (50) round.

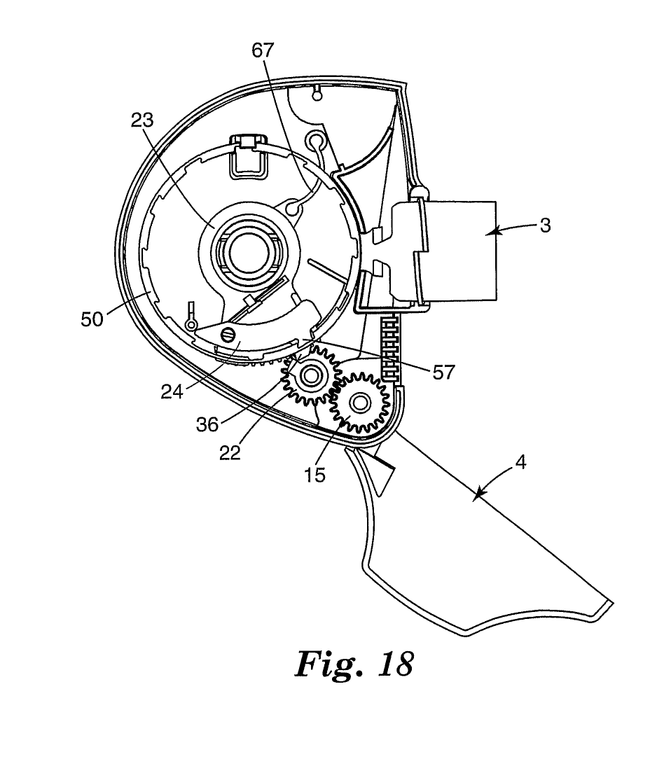

Referring to FIG. 18, as the outlet cover (4) is continued to be opened, further rotation of the gears (15, 22) and the indexing yoke (23) cause the indexing tooth (57) on the indexing arm (24) to pull the carousel (50) round almost to the position at which the next receptacle (not shown) is aligned with the patient-outlet (3). The bias link (67) in the meantime returns to its original curvature, thus providing some additional force in assisting the opening of the outlet cover. The bias link (67) thus provides an advantageous “positive open-positive close” feature, as it serves to bias the outlet cover (4) to either its fully open or fully closed position, thereby helping to ensure full advancement of receptacles and their correct registration with the impactor and the patient-outlet. This arrangement also provides tactile feedback to the patient that the outlet cover (4) has been correctly opened or closed. It also prevents the outlet cover (4) from flapping loosely when fully opened.

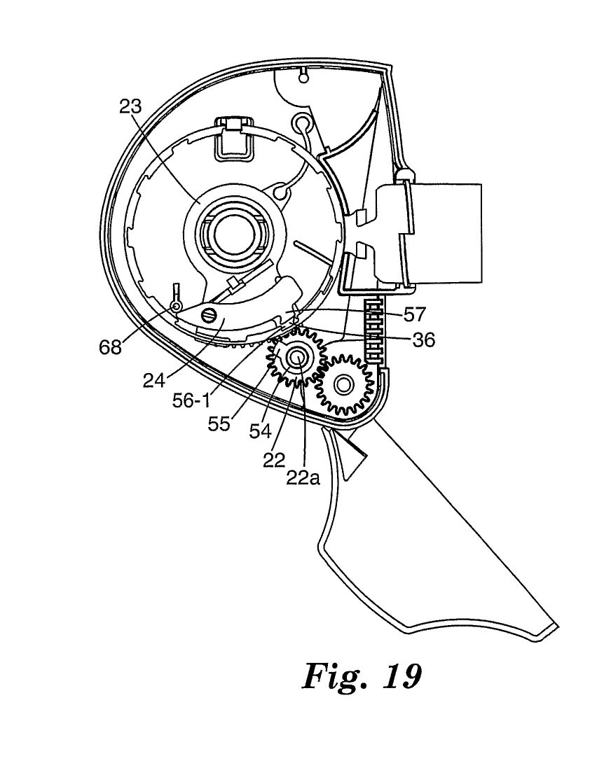

Referring to FIG. 19, as the outlet cover is opened to its fully opened position, additional rotation of the indexing yoke (23) causes the nose of the indexing arm (24) to collide with a stop peg (68) molded onto the internal surface of the body, thereby causing the indexing arm (24) to rotate counter-clockwise (as shown in FIG. 19), against the force of the torsion spring (25, not shown in FIG. 19). The rotation of the indexing arm (24) causes its indexing tooth (57) to disengage from the carousel advancement recess (56-1) that it has been driving. The carousel (50) is now positioned ready for inhalation, and the device is ready for breath-actuation by the patient. This arrangement provides for the to-be-fired receptacle to be positioned ready, for perforation, in advance of the start of inhalation, thereby ensuring that receptacle advancement imposes no delay on, and takes no energy from, the triggering mechanism.

The patient at this point in time may decide not to use the device and close the outlet cover. In this case devices described herein advantageously provide an advancing mechanism in which the next receptacle to be fired is not advanced until the previous receptacle has been fired. In particular the advancing mechanism is configured and coupled to the outlet cover such that if the receptacle advanced upon opening of the outlet cover has not been actuated (fired), then upon closing the outlet cover said receptacle is returned to its original pre-advanced position.

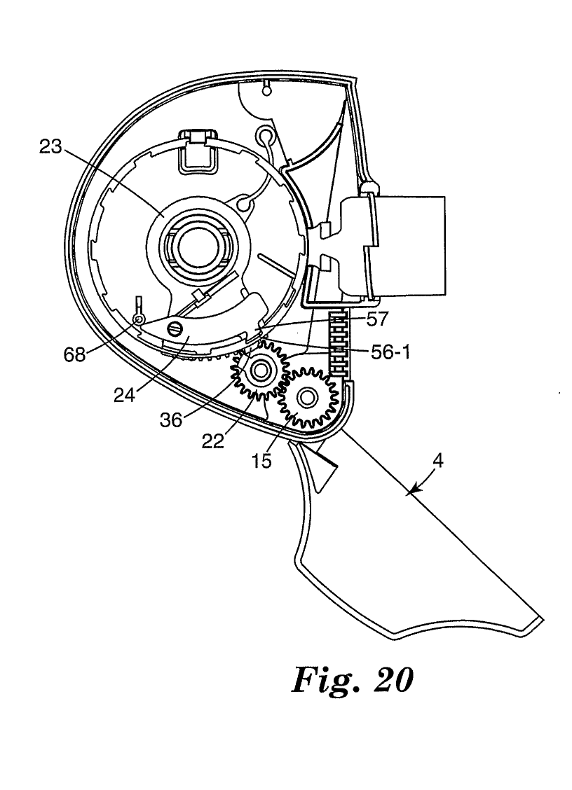

In particular FIG. 20 schematically shows the interior of the device in the case that the device has not been used (i.e. there has been no breath actuation of the device and thus the receptacle has not been actuated), but the patient has started to re-close the outlet cover. (In FIG. 20, the outlet cover (4) has been closed to a position of about 125°.) As the patient begins to close the outlet cover (4), this causes a reverse rotation of the gears (15, 22). This counter rotation in turn causes a reverse rotation of the indexing yoke (23), thereby allowing the indexing arm (24) to move off the stop peg (68) and thus allowing the indexing tooth (57) to start to re-engage with the same carousel advancement recess (56-1) that it has previously driven. Further closure of the outlet cover (4) then reverses the sequence of events that occurred during the opening of the cover, and the carousel (50) is thus returned to its original position. The bias link (67) helps to ensure that the outlet cover (4) is returned to its fully closed position. The provision of an advancing mechanism allowing for a reversal in advancement when a patient simply opens and closes the outlet cover without actually using the device (i.e. without actuating the receptacle advanced during the opening of the cover) ensures that no receptacle is wasted. This is particularly critical when the receptacle contains expensive pharmaceutically active ingredient(s).

In the event that the patient has used the device (e.g. has through breath actuation actuated the device and thus fired the advanced receptacle), the advancing mechanism is configured such that upon closing the outlet cover, the expended receptacle remains in the registered position and then upon a sequential opening of the outlet cover, the next receptacle to-be-fired is then advanced to the registered position.

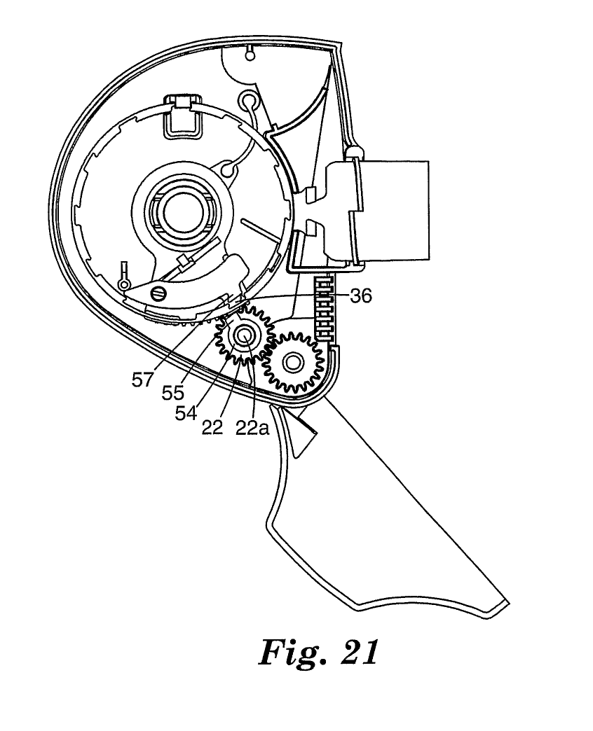

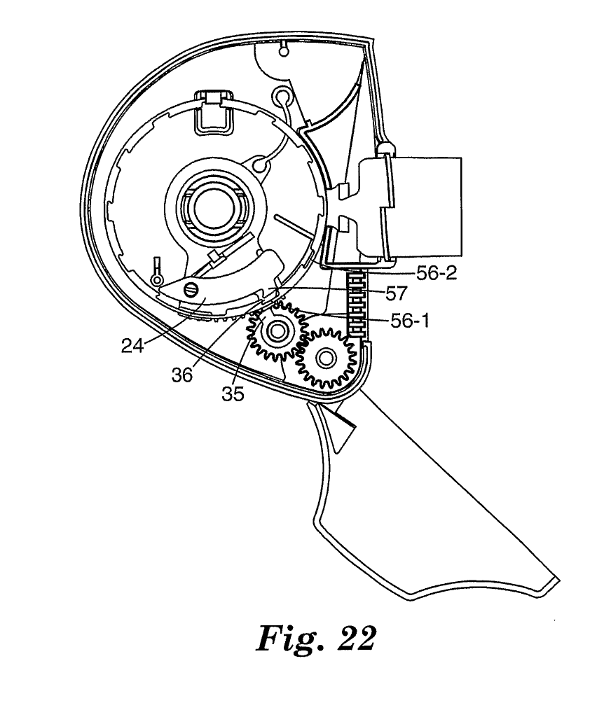

Referring to the exemplary embodiment, this is best understood by comparing FIGS. 19 and 21 showing interior views before and after actuation (firing of the receptacle) and FIGS. 22 to 24 showing views upon closing the outlet cover after actuation of the device.

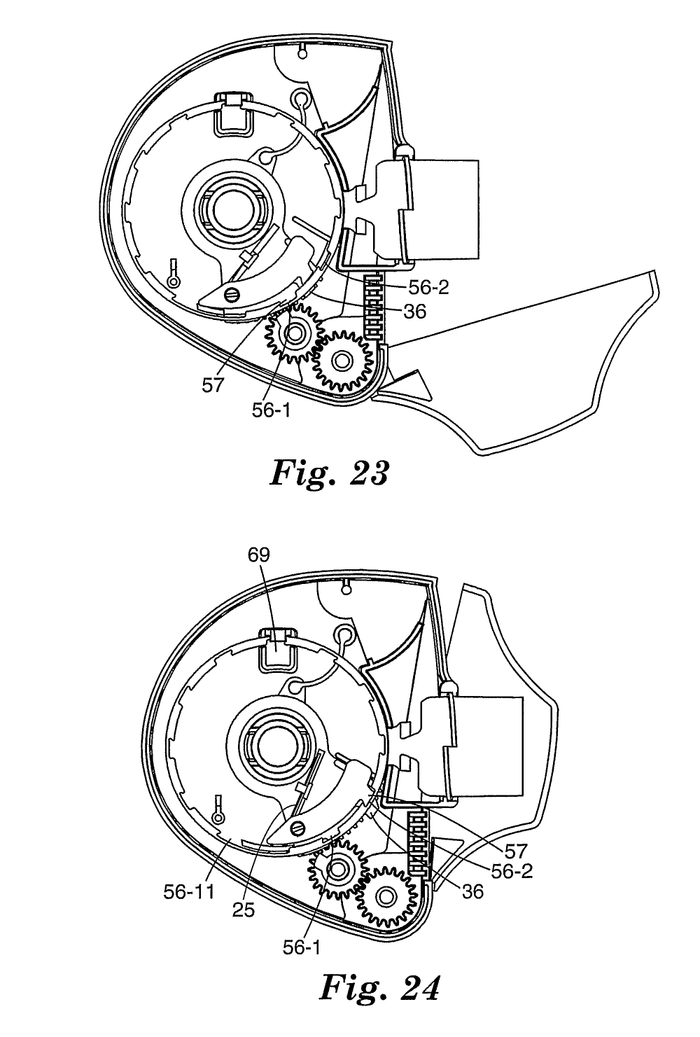

Referring to FIG. 19, the device is ready for actuation and the indexing tooth (57) of the indexing arm (24) is disengaged from the carousel advancement recess (56-1). Also referring to FIG. 19, it can be noted that the rotation of the indexing arm against the force of the torsion spring causing the disengagement of the indexing tooth (57) from the recess (56-1) also causes a corresponding movement of the pawl (36) of the indexing arm (24) away from the impactor shaft cam (55). FIG. 21 shows a view after actuation of the device allowing the triggering mechanism to fire the device, in particular allowing the impactor to rotate and thus strike the base of the receptacle to displace the receptacle to the firing pin for perforation and thus release of the dose (as discussed in detail in connection with the first exemplary embodiment). In comparison to the state before actuation (FIG. 19) it can be seen that the rotation of the impactor (13, not completely shown) for firing has also caused the impactor shaft cam (55) to rotate (clockwise, as shown in FIG. 21), into a position where the cam (55), in particular the end of the cam, obstructs the return path of the indexing arm pawl (36) and thus any re-engagement of the indexing tooth (57) with the same carousel advancement recess (56-1) that the indexing tooth has previously driven. Now—referring to FIG. 22—as the patient beings to close the outlet cover (4) (the cover has been closed to a position of 1250 in FIG. 22), the end of the indexing arm pawl (36) is forced to rise over the impactor shaft cam (55), thus holding the indexing arm (24) (in its counter-clockwise position in FIG. 22) against the bias force of its torsion spring (not shown in FIG. 22). As the patient continues to close the outlet cover (4) the indexing arm continues to move around (counter-clockwise in FIGS. 23 and 24) relative to the carousel (50) until the indexing tooth (57) enters the next carousel advancement recess (56-2) with which it engages under the action of the torsion spring (25) (as shown in FIG. 24 wherein the cover is almost completely closed). When the outlet cover is again opened, the next carousel advancement recess (56-2), and thus the next receptacle to-be-fired, will be driven round to the registered position.

In FIG. 24, it can be seen that the last two carousel advancement recesses are effectively joined together as one single long recess (56-11). By this arrangement, the carousel (50) can be prevented from rotating more than one complete turn (i.e. to previously used receptacles): the indexing tooth (57) can never leave the long carousel advancement recess (56-11) once it reaches it. Provision of such a feature reduces the number of accessible doses to eleven in this embodiment, however. This long recess (56-11) could be used during assembly to ensure correct orientation of the carousel (50) in the body (2). Also the carousel may be provided with the appropriate dose count indicia made visible to the patient through an appropriate window (69) in the body (2). The window may be sealed (e.g. with a transparent or locally transparent label) if desired.

FIG. 25 provides an exploded diagram of the indexing yoke (23), indexing arm (24), torsion spring (25), and bias link (67) used in the second exemplary embodiment.

As will appreciated by the skilled reader the first exemplary embodiment shown in FIGS. 1 to 13 includes the same advancing components and arrangement as the second exemplary embodiment shown in FIGS. 14 to 25, with the exception of the presence of a bias link, and thus the dose advancing mechanism of the first exemplary embodiment operates in a similar manner to that of the second exemplary embodiment. The first exemplary embodiment may also be provided with a curved bias link (67) similar to that shown in the second exemplary embodiment, providing a third exemplary embodiment. FIGS. 28a and b show interior views of (just) the advancing mechanism of such an embodiment showing the positions of the mechanism when the outlet cover (4) is closed and opened, respectively. As can be seen, the bias link (67) may be anchored to a point (67a) on the internal surface of the outer housing (16) of the cassette (5) and to a point (67b) on an extension (23a) provided on the indexing yoke (23). In this case the advancing mechanism operates in the same manner as described for the second exemplary embodiment. It is to be understood that the description concerning dose advancement in the second exemplary embodiment applies appropriately or completely to the first and third exemplary embodiments, respectively. Also as shown in FIGS. 28a and b, the third embodiment (as well as the first embodiment) may also include a window (69) in the outer casing (16) of the cassette (5) allowing the patient to view the carousel and any appropriate dose count indicia provided on the carousel.

The above description provides but three examples of embodiments of an inhalation device. Alternative embodiments of this invention may be envisaged. As particular examples, the axis of rotation of the carousel may be parallel to the direction of impaction of the receptacles rather than perpendicular to it, and/or the impactor head (comprising a circular impaction ring with a through-hole in it) may strike a firing pin, which may be re-usably part of the impactor or may be associated with the individual receptacle, against the front (dose release) side of the receptacle, spray thereby passing through the hole in the impactor head. Alternative and/or additional features may be envisaged, such as the provision of features in the outlet region including baffles, as disclosed in EP 551 338 (to McAughey and Pritchard), or a large bowl arrangement, as disclosed in U.S. Pat. No. 5,115,803 (to Sioutas).

Inhalation devices described herein comprise two or more hermetically sealed receptacles. Devices described herein are particularly suitable for containing three or more, more particularly four or more and most particularly five or more hermetically sealed receptacles. The total number of receptacles provided within the device depends in part of the particular targeted use of the device and in part on size considerations, for example the dimensions of each individual receptacle and the dimensions of the inhalation device. In regard to the latter considerations, preferable the inhalation device is a hand-held device. Devices described herein will typically include 30 individual receptacles at most. In regard to the former consideration (targeted use) the total number of receptacles may be appropriately selected for the particular therapy. For example for a particular therapy requiring one dose of medicament per day over a period of a week, 10 days, two weeks or three weeks, the device may appropriately include seven, ten, fourteen or twenty one receptacles, respectively.

Devices may be refillable. For example the device may include an access panel whereby the patient or a pharmacist can refill the device with fresh receptacles (and for preferred embodiments their associated firing pins). More desirably as mentioned above, devices may be provided as a kit-of-parts including a body portion and a cassette comprising receptacles (and in preferred embodiments their associated firing pins) and optionally one or more such cassettes as replacement cassettes. The body portion would desirably include a patient outlet (favorably a reversibly detachable outlet for washing) and/or a triggering mechanism and/or if applicable an outlet cover, e.g. as described herein. Such a kit may for example include the appropriate number of cassettes (and thus receptacles) for a particular therapy. For example for a 3-week therapy of two doses per day, the kit may include 3 cassettes each with 14 receptacles. Alternatively e.g. for long-term therapies, such cassettes may be provided separately, so that a patient may obtain e.g. through the pharmacist, fresh cassettes as needed for re-filling.

The pressurized formulation contained within each receptacle and comprising a pharmaceutically active ingredient and a liquefied aerosol propellant consisting of 1,1,1,2-tetrafluoroethane (HFA 134a), 1,1,1,2,3,3,3-heptafluoropropane (HFA 227) or a mixture thereof may optionally comprise one or more suitable pharmaceutical non-propellant and non-gaseous excipients, such as surfactants, preservatives, flavorings, antioxidants, anti-aggregating agents and co-solvents, e.g. ethanol. The term “excipients” as used herein means chemical agents having little or no pharmacological activity (for the quantities used) but which may enhance the pharmaceutical formulation or the performance of the inhalation device. Under the term “non-propellant excipient” is to be understood that the excipient is not a propellant. “Propellant” used herein means an inert liquid with a boiling point from about 25° C. to −43° C. which exerts a high vapor pressure at room temperature, e.g. a hydrocarbon (such as propane, butane, isobutane), a chlorofluorocarbon or a hydrogenated-chlorofluorocarbon. Under the term “non-gaseous” excipient is to be understood that the excipient is not a gas (i.e. a substance having a boiling point less than −43° C., e.g. carbon dioxide, oxygen, nitrogen).

It will be appreciated by those skilled in the art that the pharmaceutical pressurized formulation for use in the invention may contain a single pharmaceutically active ingredient or a combination two or more other pharmaceutically active ingredients.

Such pharmaceutically active ingredients may be selected from any suitable medicaments used in inhalation therapy. Appropriate medicaments may thus be selected from, for example,
analgesics, e.g. codeine, dihydromorphine, ergotamine, fentanyl or morphine;
anginal preparations, e.g. diltiazem, nitroglycerin;
antiallergics, e.g. cromoglycate, ketotifen or nedocromil;
antiinfectives e.g. cephalosporins, penicillins, streptomycin, sulphonamides, tetracyclines and pentamidine;
antihistamines, e.g. methapyrilene;
anti-inflammatories, e.g. corticosteroids, such as beclomethasone (e.g. the dipropionate), betamethasone, flunisolide, budesonide, ciclesonide, mometasone (e.g. the fuorate), fluticasone (e.g. the propionate). triamcinolone acetonide, cortisone, dexamethasone, hydrocortisone, methylprednisolone, prednisolone or prednisone;
antitussives, e.g. noscapine;
bronchodilators, e.g. salbutamol, salmeterol, ephedrine, adrenaline, fenoterol, formoterol, isoprenaline, metaproterenol, phenylephrine, phenylpropanolamine, pirbuterol, reproterol, rimiterol, terbutaline, isoetharine, tulobuterol, orciprenaline, or (−)-4-amino-3,5-dichloro-alpha-[[[6-[2-(2-pyridinyl)ethoxy]hexyllamino]-methyl]benzenemethanol;
diuretics, e.g. amiloride;
antimuscarinics, e.g. anticholinergics such as ipratropium, atropine or oxitropium;
xanthines e.g. aminophylline, choline theophyllinate, lysine theophyllinate or theophylline;
phosphodiesterase inhibitors, e.g. PDE-4 inhibitors such as roflumilast; and leukotriene modifiers, e.g. montelukast or zafirlukast. It will be clear to the person skilled in the art that where appropriate, the medicaments may be used as a free base or in a physiologically acceptable form, e.g. in the form of salts (e.g. as alkali metal or amine salts or as acid addition salts) or as esters (e.g. lower alkyl esters) or as solvates (e.g. hydrates).

Inhalation devices in accordance with the present invention are advantageous for use in providing a plurality of single doses of very expensive and/or very sensitive pharmaceutically active ingredients cost-effectively, e.g. especially for therapies including macromolecules (e.g. proteins and peptides) or other biological products. Accordingly, the pharmaceutically active ingredient may advantageously be selected from insulin, glucagon, g-csf (granulite colony simulating factor), erythropoietin, growth hormone, alpha-interferon, beta-interferon, calcitonin, alpha-1-anti-trypsin, oxytosin, somatostatin, parathyroid hormone, tnf (tumor-necrosis-factor)-alpha, Dnase, vasopressins (e.g. arginine vasopressin and ornithine vasopressin), LHRH analog, bovine-IgG, ferritin, gene transfer or therapy preparations (e.g. recombinant vectors (viral or non-viral), virus, naked or complex plasmids, virus producing cells, in vitro genetically modified cells, or portions of nucleic acids (e.g. for anti-sense type therapy)), somatic cell therapy preparations, molecules issued from rDNA and vaccines.

Devices described herein are particularly advantageous in that each receptacle may contain a different pharmaceutical active ingredient or combination of pharmaceutical active ingredients. For example, a device may include four receptacles, each containing a different vaccine formulation, so that a single inhalation device (e.g. a disposable inhalation device) may be used to administer to a patient sequentially four different vaccines.

Because the devices described herein advantageously allow actuation of the receptacles contained therein in sequence, the devices can be particularly useful for therapies in which the dosage of a pharmaceutically active ingredient is tapered down (e.g. anti-infectives/antibiotics) or ramped up during therapy. In other words, the dosage of the pharmaceutically active ingredient in one receptacle may be either higher or lower (as desired or needed) than the dosage of the pharmaceutically active ingredient in another receptacle. For devices comprising three or more (more suitably four or most suitably five or more receptacles), the receptacles may be provided in a prescribed order, wherein the dosage of pharmaceutically active ingredient in said receptacles in said prescribed order decreases continuously or intermediately (e.g. stepwise) or decreases to a constant plateau. Alternatively the dosage of pharmaceutically active ingredient in said receptacles in said prescribed order may increase continuously or intermediately (e.g. stepwise) or increase to a constant plateau. Through such devices the desired dosage with the appropriate tapering down or ramping up of said dosage is automatically administered to the patient.

Devices described herein are also particularly advantageous for therapies administrating two or more pharmaceutically active ingredients sequentially, e.g. where one active ingredient is desirably administered before another active ingredient (e.g. a bronchodilator administered shortly before an anti-inflammatory, to assure that the anti-inflammatory reaches its target site in the lungs) or where the selected active ingredients can be not be combined in a single formulation. In such sequential combination therapies with e.g. two active ingredients, the device advantageously comprises an even number of receptacles (e.g. 2n where n is 1, 2, 3, 4, 5, 6, 7, 8, 9, 10, 11, 12, 13, 14 or 15), wherein the odd numbered receptacles in the sequence (e.g. 1^{st} to (2n−1)^{th} receptacle) each contain a first formulation comprising a first active ingredient and the even numbered receptacles in the sequence (e.g. 2^{nd} to (2n)^{th} receptacle) contain a second formulation comprising a second active ingredient. One active ingredient may be a bronchodilator, a xanthine or a leukotriene modifier, while the other active ingredient may be an anti-inflammatory, in particular a corticosteroid. For the combinations, a bronchodilator and an anti-inflammatory (in particular a corticosteroid) or a xanthine and an anti-inflammatory (in particular a corticosteroid), preferably the anti-inflammatory is the second active ingredient (i.e. the anti-inflammatory is to be administered after the bronchodilator or xanthine). For the combination a leukotriene modifier and an anti-inflammatory, the anti-inflammatory can be either the first or second active ingredient. The first and/or second formulations may comprise additional active ingredients as desired or needed. For example, it may be advantageous to provide a three medicament combination therapy with a bronchodilator and an anti-inflammatory (in particular a corticosteroid) together with a leukotriene modifier, wherein the leukotriene modifier is formulated with either the bronchodilator or the anti-inflammatory, preferably with the anti-inflammatory, and more preferably with the bronchodilator being the first active ingredient (i.e. to be administered before the anti-inflammatory and leukotriene modifier). Alternatively it may be favorable to administer three medicaments (e.g. the aforementioned bronchodilator, leukotriene modifier and anti-inflammatory) separately one after another. In this case the device may include 3n number of receptacles (e.g. where n is an integer greater than or equal to 1, in particular n is 1, 2, 3, 4, 5, 6, 7, 8, 9 or 10) and wherein the first receptacle in each sequence of three contains a first active ingredient, the second receptacle in each sequence of three contains second active ingredient and the third receptacle in each sequence of three contains a third active ingredient. Referring to bronchodilator, leukotriene modifier, anti-inflammatory combination, preferably the bronchodilator is the first active ingredient.

1. An inhalation device comprising two or more hermetically sealed receptacles, each receptacle containing a single dose of a pressurized formulation comprising a pharmaceutically active ingredient and a liquefied aerosol propellant consisting of HFA 134a, HFA 227 or a mixture thereof and wherein at least a portion of each receptacle is perforable.

2. An inhalation device according to claim 1, wherein the device further comprises a tiring pin and a triggering, mechanism, said firing pin comprises a channel having, an opening at both ends, wherein the device is arranged such that upon actuation of the device the triggering mechanism is released and acts on a receptacle and/or the firing pin so that the receptacle and the firing pin are mutually displaced to cause the firing pin to perforate the receptacle at said at least perforable portion and to cause aerosol formulation to pass through said channel from the first end to the second end of the channel and through to the patient.

3. An inhalation device according to claim 2, wherein the device is arranged, such that upon actuation of the device the receptacle is displaced towards the firing pin, which is held fixed relative to the device, to cause the firing pin to perforate the receptacle at said at least perforable portion and to cause aerosol formulation to pass through said channel from the first end of the second end of the channel and through to the patient.

4. An inhalation device according to claim 3, wherein the triggering mechanism comprises a mechanically or pneumatically loaded impactor that is released upon actuation and acts on the receptacle displacing it towards the firing pin.

5. An inhalation device according to claim 2, wherein the device is arranged such that upon perforation of said at least perforable portion of the receptacle by the firing pin the first end of the channel of the firing pin passes into a liquid portion of the pressurized formulation.

6. An inhalation device according to claim 2, wherein the firing pin has an internal surface and the internal surface defines the channel.

7. An inhalation device according to claim 6, wherein the internal surface of the firing pin defining the channel is arranged as to provide an expansion chamber.

8. An inhalation device according to claim 6, wherein the internal surface of the firing pin defining the channel is generally conical from the first end to the second end.

9. An inhalation device according to claim 2, wherein the outermost surface of the firing pin is provided with a piercer capable of perforating said at least perforable portion of the receptacle and wherein the first end of the channel of the firing pin is set back from said outermost surface and positioned adjacent to said piercer.

10. An inhalation device according to claim 2, wherein the outer surface of the portion of the firing pin that passes into the receptacle upon perforation of said at least perforable portion of the receptacle is conical in shape.

11. (canceled)

12. (canceled)

13. (canceled)

14. An inhalation device according to claim 1, wherein the device comprises two or more firing pins, wherein each receptacle is in operative association with an individual firing pin.

15. An inhalation device according to claim 1, wherein the device further comprises a carrier wherein the receptacles are mounted on the carrier.

16. An inhalation device according to claim 15, wherein a portion of the device comprising the carrier and mounted receptacles is reversibly removable from the device.

17. An inhalation device according to claim 16, wherein said portion is provided in the form of a cassette and the device is provided as a kit-of-parts comprising a body portion and at least one cassette.

18. An inhalation device according to claim 16, wherein said portion comprising the carrier and mounted receptacles further comprises the firing pins with which the receptacles are in operative association.

19. (canceled)

20. (canceled)

21. (canceled)

22. (canceled)

23. (canceled)

24. (canceled)

25. (canceled)

26. (canceled)

27. An inhalation device according to claim 1, wherein the internal volume of each receptacle is less than 0.3 ml.

28. (canceled)

29. (canceled)

30. (canceled)

31. An inhalation device according to claim 1, wherein each receptacle is free of elastomeric seals and diaphragms and/or dispensing valves.

32. (canceled)

33. An inhalation device according to claim 1, wherein the said at least perforable portion is substantially planar.

34. An inhalation device according to claim 1, wherein the at least perforable portion has a thickness of 250 μm at most.

35. (canceled)

36. (canceled)

37. (canceled)

38. (canceled)

39. (canceled)

40. (canceled)

41. An inhalation device according to claim 1, the at least perforable portion is a foil.

42. An inhalation device according to claim 41, wherein the foil is laser welded to form a hermetic seal.

43. (canceled)

44. (canceled)

45. (canceled)

46. (canceled)

47. (canceled)

48. An inhalation device according to claim 1, wherein the device comprises even number of receptacles, wherein the odd numbered receptacles in the sequence contain a first active ingredient and the even numbered receptacles in the sequence contain a second active ingredient.

49. (canceled)

50. (canceled)

51. (canceled)

52. (canceled)

53. An inhalation device according to claim 1, wherein the device comprises 3n number of receptacles where n is an integer equal to or greater than 1, and wherein in each sequence of three receptacles, the first receptacle contains a first active ingredient, the second receptacle contains a second active ingredient and the third receptacle contains a third active ingredient.

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)
